(19) United States
(12) Patent Application Publication
(10) Pub. No.: US 2017/0224220 A1
(43) Pub. Date: Aug. 10, 2017

(54) MULTI-MODAL FIBER OPTIC PROBE AND SPECTROSCOPY SYSTEM

(71) Applicant: BOARD OF REGENTS, THE UNIVERSITY OF TEXAS SYSTEM, Austin, TX (US)

(72) Inventors: James W. Tunnell, Austin, TX (US); Manu Sharma, Viejo, CA (US); Eric Marple, Loxahatchee, FL (US)

(21) Appl. No.: 15/502,023

(22) PCT Filed: Aug. 4, 2015
(86) PCT No.: PCT/US15/43664

§ 371 Date: Feb. 6, 2017

Related U.S. Application Data
(60) Provisional application No. 62/033,098, filed on Aug. 4, 2014.

(51) Int. Cl. A61B 5/00; G01J 3/02; G01J 3/44

(52) U.S. Cl. CPC A61B 5/0071; G01J 3/4406; G01J 3/4412; G01J 3/0218; A61B 5/725; A61B 5/444; A61B 5/0064; A61B 5/0075

(57) ABSTRACT

A fiber-optic probe for multi-modal characterization of a tissue. The fiber optic probe may comprise a first group of fibers associated with a first modality of light. The first group of fibers may comprise a first light delivery fiber and a first light collection fiber. The fiber optic probe may also comprise a second group of fibers associated with at least a second modality of light, the second group of fibers comprising a second light delivery fiber and a second light collection fiber; The fiber optic probe may also comprise a longpass filter positioned distal to the first group of fibers and a lens positioned distal to the filter. The fiber optic probe may also comprise. The fiber optic probe may include the second group of fibers bypassing the filter. The second group of fibers may bypass the filter.

This application claims benefit of U.S. Provisional Application No. 62/033,098, filed Aug. 4, 2014, which is hereby incorporated herein by reference in its entirety.

### TECHNICAL FIELD

The present disclosure relates generally to systems and methods for multi-modal characterization of biological tissue and, more particularly, to methods for detecting cutaneous lesions and corresponding multimodal fiber-optic probe and spectroscopy system.

### BACKGROUND

Spectroscopic techniques utilize the interaction of light with biological tissue to study tissue optical properties, which change with disease progression and can be used for diagnosis. Deployment of spectroscopic-based devices has the potential to significantly augment clinical diagnosis. Three common spectroscopic techniques are Raman spectroscopy (RS), diffuse reflectance spectroscopy (DRS), and laser-induced fluorescence spectroscopy (LIFS or LFS). These techniques have been applied—either individually or in combinations—throughout the entire human body to investigate a wide range of pathologies including: atherosclerosis, osteoporosis, brain edema, cataract formation, kidney stones, diabetes, and cancer of the breast, cervix, esophagus, gastrointestinal tract, brain, lungs, ovaries, and bladder. Here, we present a novel multi-modal spectroscopy (MMS) device, combining RS, LIFS, and DRS, for the purpose of fast and non-invasive early detection of skin cancer which uses a variety of instrumentation and a custom contact probe capable of delivering and collecting light for all three modalities.

Skin cancer, in both its melanoma and non-melanoma forms, has the largest reported incidence of all cancers in the United States. North America has the 2nd highest age-standardized melanoma incidence rate in the world. Early identification of skin cancer is paramount for its effect upon patient survival; the five-year “regional” survival rate of melanoma is 62%, dropping to 16% for “distant” detection. Significant ambiguity exists for the clinical distinction via visual inspection between dysplastic nevi and melanoma, raising issues of considerable practical and financial importance. Currently, the only reliable method of distinguishing between dysplastic nevi and melanoma is via stained biopsies, which is invasive and expensive. Clinician specificity for melanoma is approximately 4%, which means that roughly 25 more times biopsies are performed than required, translating to an estimated cost of $6×10⁹ to the US health care system. In Q2 2007, the mean wait time for new patients in urban areas to see a dermatologist was 33 days; in rural areas, the number rises to 46 days. Furthermore, delay in diagnosis due to “off site” analysis imparts an emotional cost to the patient.

Skin cancer patient care is currently limited by the invasive nature of biopsies and the high cost associated with biopsies due to inadequate screening techniques. Motivated by improving this degree of patient care, we present a noninvasive, real-time spectroscopic-based technology to significantly improve lesion pathology specificity for the early detection of skin cancer. Ultimately, this device will save lives through early detection and its improved differentiation between skin pathologies will translate directly to lower costs and morbidity.

In short, MMS characterizes the tissue microenvironment via morphological changes observed through DRS and bio-chemical information via RS and LIFS. The DRS measurement is a function of tissue scattering and absorption properties, which in turn are dependent upon tissue morphological changes. Hence, analysis yields information about tissue blood fraction, oxygen saturation, tissue scattering coefficient, nuclear morphology, and collagen structure. LIFS is biochemically sensitive as it interrogates endogenous fluorophores such as nicotinamide adenine dinucleotide (NADH), flavin adenine dinucleotide (FAD), and collagen. Their fluorescence levels change with neoplastic progression that is associated with altered cellular metabolic pathways (NADH, FAD) or an altered structural tissue matrix (collagen). Raman spectroscopy exploits the inelastic scattering (so-called “Raman” scattering) phenomena to detect spectral signatures of important disease progression biomarkers, including lipids, proteins, and amino acids. Raman spectroscopy is more constituent-specific than fluorescence and is capable of spectrally “breaking down” the biochemical composition; however, the two techniques are complementary as they probe different bio-molecular species.

As different techniques provide complementary information, it has been shown that an instrument combining multiple techniques offers a more precise description of disease status. Specifically for skin cancer, it has been demonstrated that a modal combination improves malignant melanoma and non-melanoma (basal and squamous cell carcinomas) diagnosis. Furthermore, MMS has been successfully applied for the early detection of atherosclerotic plaque. The successful application of MMS in non-cancer related pathologies indicates that it has considerable potential and its efficacy would be further tested by applying it to skin cancer.

Biomedical optical spectroscopy measurements very commonly use fiber-optic probes, which serve as the optical interface between the sample and the spectroscopic equipment. The fiber-optic probes contain fiber bundles that are responsible for both delivering and collecting light from the sample. Many types of probes have been used within the research community and cannot all be summarized here; however, comprehensive reviews of DRS, LIFS, and RS probe technology are available. Briefly, combination LIFS-DRS probes have been demonstrated by past studies, as well as a probe combining all three techniques.

There are several important, and challenging, design and functional considerations for a probe that combines reflectance, fluorescence, and Raman signals. First, due to the very low probability of Raman scattering (one inelastically scattered photon for every elastically scattered photons), the optical design must be fine-tuned to maximize RS signal-to-noise ratios. Second, the traditional source-detector geometry employed for reflectance measurements needs to be preserved such that tissue scattering and absorption properties can be separated and quantified.

The presently disclosed systems and methods for multi-modal characterization of biological tissue are directed to overcoming one or more of the problems set forth above and/or other problems in the art.

### SUMMARY

According to one aspect, the present disclosure is directed to a method for multi-modal characterization of biological tissue. The method may comprise delivering, via a first optical fiber through a first transmission medium, first light from a first light source onto a biological tissue. The method may also comprise collecting, via a second optical fiber through the first transmission medium, light emitted from the biological tissue in response to the first light. Second light may be delivered, via a third optical fiber through a second transmission medium, from a second light source onto the biological tissue. The method may also comprise collecting, via a fourth optical fiber through the second transmission medium, light emitted from the biological tissue in response to the second light. The method may further comprise processing the light collected by the second and fourth optical fibers to determine a characteristic of the biological tissue.

In accordance with another aspect, the present disclosure is directed to a method for multi-modal characterization of biological tissue. The method may comprise collecting first light emitted from a biological tissue through a first transmission medium of a fiber-optic probe, the first transmission medium including a lens. The method may also comprise collecting second light emitted from the biological tissue through a second transmission medium, the second transmission medium bypassing the lens. The method may further comprise processing the collected first and second light to determine a characteristic of the biological tissue.

According to another aspect, the present disclosure is directed to a fiber-optic probe for multi-modal characterization of a tissue. The fiber optic probe may comprise a first group of fibers associated with a first modality of light. The first group of fibers may comprise a first light delivery fiber and a first light collection fiber. The fiber optic probe may also comprise a second group of fibers associated with at least a second modality of light, the second group of fibers comprising a second light delivery fiber and a second light collection fiber; The fiber optic probe may also comprise a longpass filter positioned distal to the first group of fibers and a lens positioned distal to the filter. The fiber optic probe may also comprise. The fiber optic probe may include the second group of fibers bypassing the filter. The second group of fibers may bypass the filter.

### BRIEF DESCRIPTION OF THE DRAWINGS

FIG. 1A provides a schematic diagram illustrating a distal portion of a fiber-optic probe, consistent with certain disclosed embodiments.

FIG. 1B provides a schematic diagram illustrating an assembly exploded view with optical elements such as the filters and front lens identified along with the collection and delivery fibers for all three modalities, in accordance with certain disclosed embodiments.

FIG. 2 provides an illustration of the MMS clinical system (left) and the MMS probe (right).

FIG. 3 illustrates a comparison between fitted (LUT) and expected (experimental) for the reduced scattering coefficient (left) and absorption coefficient (right).

FIG. 4 illustrates sample in vivo Raman spectra obtained using the MMS probe for various body locations. Exposure time is 4 s.

FIG. 5 illustrates MMS clinical data of a basal cell carcinoma for Raman (left), diffuse reflectance (right), and intrinsic fluorescence (right) modalities. Physiological quantities obtained from DRS spectral fitting were μ′s (λ₀=1.7 mm⁻¹, ν=2.87, α=0.46, and Dᵥₑₛₛₑₗ=0.48 μm and χ_{nadh}=0.55 and χ_{collagen}=0.45 from the intrinsic fluorescence spectral fitting.

FIG. 6A illustrates a schematic diagram illustrating an assembly exploded view with optical elements such as the filters and front lens identified along with the collection and delivery fibers for all three modalities, in accordance with certain disclosed embodiments.

FIG. 6B a schematic diagram illustrating a distal portion of a fiber-optic probe, consistent with certain disclosed embodiments.

FIG. 7 illustrates a schematic diagram illustrating an assembly exploded view with optical elements such as the filters and front lens identified along with the collection and delivery fibers for all three modalities, in accordance with certain disclosed embodiments, and a schematic diagram illustrating a distal portion of a fiber-optic probe, consistent with certain disclosed embodiments

### DETAILED DESCRIPTION

MMS systems consistent with the disclosed embodiments are optionally modular, comprising 2 main “sub-units”: hardware to control the LIFS and the DRS measurements and separate hardware for RS acquisition. An example MMS system includes a plurality of subcomponents that cooperate to characterize the biological tissue under test. In one embodiment, an MMS system may comprise one or more light sources, an MMS probe module, a photodetection module, and data acquisition hardware and software. It is contemplated that the components of MMS system listed above and described throughout are exemplary only and not intended to be limiting. The term “exemplary” is used throughout this description to mean “example.” Those skilled in the art will recognize that MMS system may include additional, fewer, and/or different components than those listed above or described throughout. For example, MMS system may also include hardware and/or software for calibrating the probe and system, as well as software for modeling the spectral data detected by the system.

Sources

The MMS system comprises a plurality of sources, each source associated with a respective modality. According to a tri-modal system consistent with certain exemplary embodiments, MMS system comprises three sources for each of the three modalities: a pulsed Xenon flash lamp (e.g., L7684, Hamamatsu Photonics, Bridgewater, N.J.), which provides broadband 375-700 nm illumination for DRS; a pulsed 337-nm nitrogen laser (NL-100, Stanford Research Systems, Sunnyvale, Calif.) to induce NADH and collagen fluorescence; and a 830-nm diode laser (Lynx, Germany) for Raman excitation. The Raman diode laser is gated by a mechanical shutter which is controlled by triggering software written in MATLAB and LabVIEW. In order to prevent second-order dispersion contaminating the reflectance spectra, the Xenon white light is first passed through a 340-nm long-pass filter (e.g., Asahi Spectra, Torrance, Calif.) and then coupled into a fiber. The Xenon lamp provides a pulse of full width half maximum (FWHM) 2.9 μs. For LIFS, the nitrogen laser and has been configured to provide approximately 160 μJ per pulse for a pulse FWHM of 3.5 ns. The LIFS signal-to-noise ratio could be increased by increasing the pulse power; however, the value of 160 μJ strikes an effective balance between sufficient signal strength and laser cartridge lifetime. The output power of the Raman diode laser can be controlled by adjusting the supplied current through the custom software; for this application, 56 mW output power (0.198 A supplied current) at 830-nm is delivered from the laser engine. The Raman laser is housed inside the Raman module, which is completely shielded by specially constructed blackened material (e.g., Thorlabs, NJ, USA) to prevent any stray light getting in or leaking out.

Probe

The multi-spectroscopy probe may be configured to be used in contact with the skin. The distal end of the probe is a polished, flat surface to ensure that the contact is as uniform as possible across the probe diameter to prevent measurement irregularities arising from gaps between the skin and probe and non-zero contact angles. The multi-spectroscopy comprises a plurality of fibers grouped to be associated with a source of light and at least one respective modality of light. As used here, “associated with” can include when a light source or modality of light be coupled to a fiber to allow propagation of the light or modality through the fiber. For example, a group of fibers may be associated with Raman laser described above. Another group of fibers may be associated with the source for DRS and/or the source for LIFS. The DRS and LIFS modalities may share the same group of fibers or they may use separate groups of fibers.

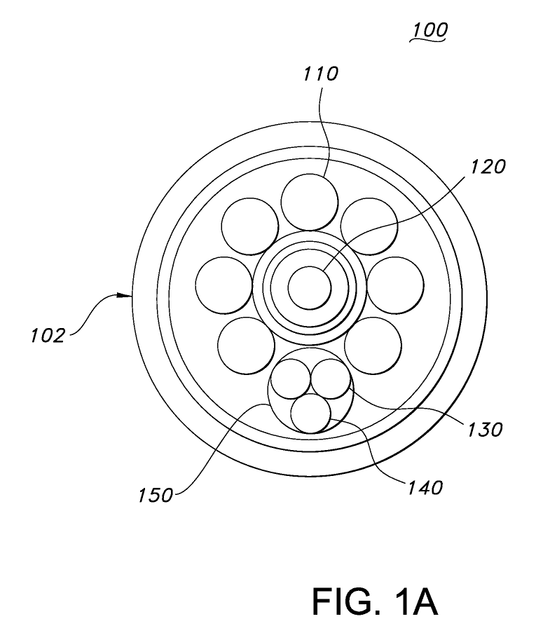

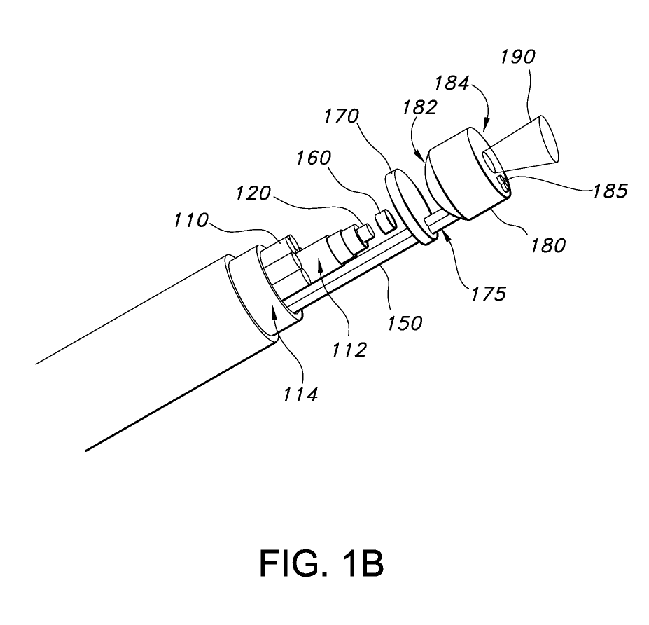

Certain embodiments of the probe design are shown in FIGS. 1, 6, and 7. In the embodiment of FIG. 1, FIG. 1A is a front-on view of the probe 100 distal end 102, and FIG. 1B is an exploded assembly view to show all the components. The fiber 120 delivering the Raman light may be centrally located at the probe distal end 102. In one example, as seen in FIG. 1A, seven 300 μm Raman collection fibers 110 and a DRS/LIFS “triangle” 150 concentrically surround a 200 μm core Raman delivery fiber 120.

The DRS/LIFS triangle 150 contains two low OH 200 μm core visible light collection fibers 130 and a high OH 200 μm core DRS/LIFS delivery fiber 140. The low and high OH cores are chosen for collection and delivery, respectively, because of the wavelength dependent attenuation characteristics of silica fiber optic cables: high OH content fibers have lower losses in the UV (hence, the selection for the LIFS delivery fiber) while low OH content fibers have lower losses in the visible (hence, the selection for the collection fibers where all the collected light is in the visible).

In certain embodiments, the triangle of fibers 150 bypass the longpass filter 170. In certain embodiments, the triangle of fibers 150 also bypasses the front lens 180. In the example embodiment shown in FIG. 1B, the triangle of fibers 150 bypasses the longpass filter 170 and the front lens 180 by passing through holes drilled in the longpass donut filter 170 and the front lens 180 as illustrated. In certain embodiments, without using some embodiment of a front lens bypass (for example, front lens bypass 185), it was seen that the source detector geometry, necessary for extracting optical properties from the reflectance signal, was not sufficiently preserved due spectral aberrations and focusing effects introduced by the front lens 180. The embodiments employing the bypass avoid these issues and allows the DRS and LIFS data to be collected in the same fashion as the standard bundle probe configuration. The tradeoff is that there is not perfect overlap between the Raman and LIFS/DRS collection spots; however, ray tracing simulations confirm that the overall delivery spot diameter (spanning all three modalities) is approx. 600 μm and this overlap is sufficient.

The Raman portion of the probe 100 uses seven low hydroxyl (OH) content 300-nm core, 0.22 NA collection fibers 110. A donut shaped 830 μm long pass filter 170 is positioned in front of these seven fibers 110, which rejects the 830 μm laser light and passes the Raman light from the sample. These seven fibers 110 surround a stainless steel tube 112 inside which is the laser delivery fiber assembly. The laser delivery fiber 120 is a 200-nm core low OH, 0.22 NA fiber which has a small 830 μm band-pass filter 160 positioned in front of it. The choice of fibers and filtering of Raman probes has been discussed by many sources previously. The two-piece converging front lens 180 is made of a plano convex 2 mm diameter curvature sapphire back portion 182 (the high refractive index bends the light sharply) and a flat front portion 184 of 1 mm thick plano Magnesium Fluoride which has virtually no Raman signature. Epoxy is used to bond the required individual components together. The fibers, lens, and other components are placed inside a stainless steel 14 gauge extra thin wall needle tube 114 (0.072 in. ID, 0.083 in. OD 2.1 mm OD).

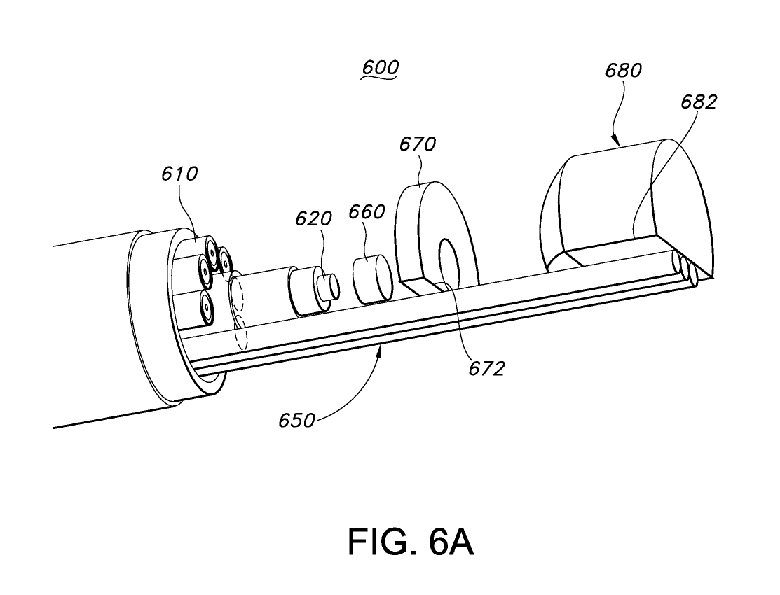

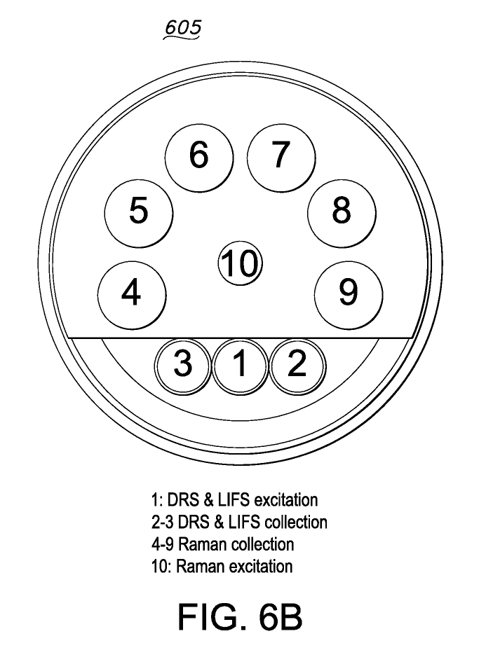

FIGS. 6A and 6B shows another embodiment of the multi-spectroscopy probe 600. FIG. 6A is an exploded assembly view to show all the components, and FIG. 6B is a front-on view of the probe distal end 605. The embodiments of FIGS. 6A and 6B includes similar features as the previous embodiment. The embodiment of FIG. 6A, however, uses an alternative to bypass the front lens and longpass filter, rather than using a hole in the filter. Here, a peripheral edge 672, 682 is formed, for example by creating a horizontal cylindrical segment, to allow the DRS/LFS fibers 650 to bypass the front lens 680 and longpass filter 670. The DRS/LFS fibers 650 at the distal end of the probe 605 can be arranged either in a triangular pattern or side-by-side. An advantage of this embodiment is that in some instances forming the peripheral edge 672, 682 can be easier and more cost effective than drilling a hole.

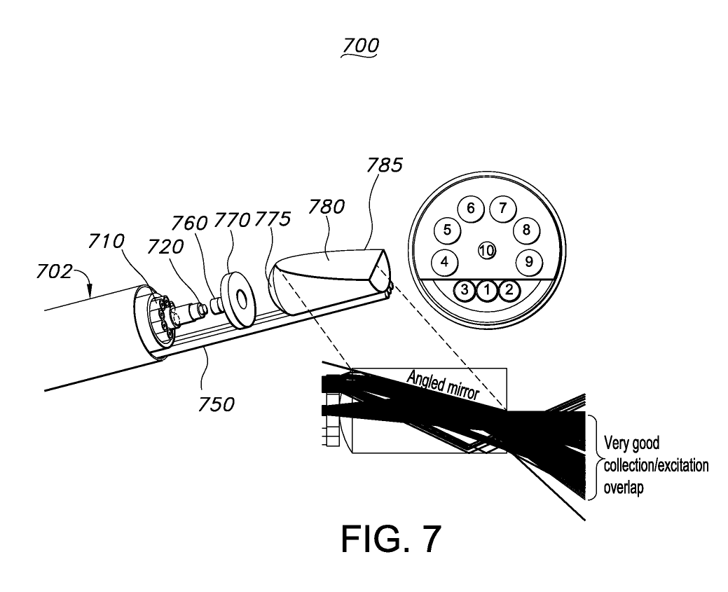

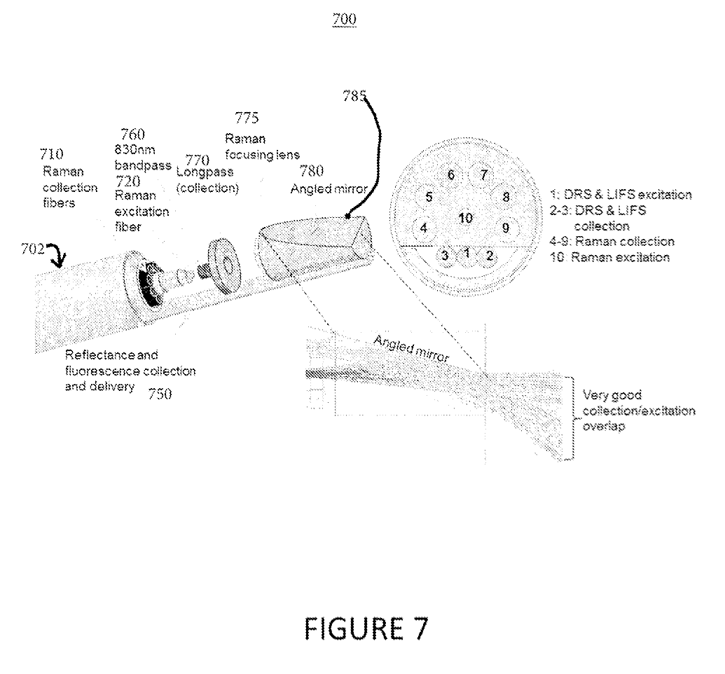

FIG. 7 shows another embodiment of the multi-spectroscopy probe 700. This embodiment comprises a wedge-shaped optic 780 (such as a mirror and/or lens) The wedge-shaped portion 785 allows the mirror/lens 780 at the most distal end of the probe 700 to have better overlap in the collection area for the Raman spectroscopy and the DRS/LFS spectroscopy. The front lens 780 is cut at an angle in a wedge shape. A reflective coating (such as a mirror) is placed onto the new surface 785 created by the wedge shape cut. The reflective coating or surface acts to redirect both the excitation and collection areas off the center axis. In this way, the sampling areas of all three spectroscopies better overlap. The DRS/LFS fibers 750 can bypass the longpass filter 770 and front lens 780 by any of the disclosed bypass mechanisms. Another example bypass mechanism, which can be combined with other embodiments, is to use a smaller diameter filter and/or lens to allow the DRS/LFS fibers to pass between the filter/lens and the casing 702 of the probe 700. An example of a smaller diameter filter and lens are show at references 770 and 750, respectively.

In another embodiment, a lookup table (LUT) algorithm is used to overcome the effects of the front lens without the use of the hole, which is used in certain other embodiments. The DRS/LFS fibers still bypass the longpass filter by one of the previously described means, such as a hole or a wedge. The DRS/LFS fibers can be arranged in a triangle or simply side-by-side. In this embodiment, the DRS/LFS fibers do not come in direct contact with the tissue surface, but rather, they are refocused through the front lens. A challenge with this arrangement is that the lens distorts the source-detector geometry of the DRS/LFS fibers at the tissue surface. Another challenge with this arrangement is that reflections from the excitation light off the front surface of the lens may propagate back to the collection fiber in the case of the DRS. The reflection issues may be minimized by using antireflection coatings at the distal end of the optics and special calibration routines. The distorted geometry of the source and collector fibers may need special algorithms to accurately measure the diffuse reflectance and determine the tissue optical properties. One such algorithm includes using look up table (LUT) approaches that account for this distortion. An advantage of this embodiment is that it avoids the need to modify the front lens with a hole or wedge. This potentially saves costs for production. Another advantage is that the DRS/LFS excitation and collection areas, although distorted, are redirected toward the center of the probe to overlap better with the Raman spectroscopy sampling spot.

Example LUT approaches are described in U.S. Patent Application Publication No. 2012/0057145, the contents of which are incorporated herein by reference in its entirety. As described in that publication, the spectra generated by the spectrophotometer may be analyzed by a look-up table (LUT) based algorithm. In certain embodiments, the LUT based algorithm is a LUT-based inverse model that is valid for fiber-based probe geometries with close source-detector separations and tissues with low albedos. In certain embodiments, the LUT inverse model may comprise (1) generating a LUT by measuring the functional form of the reflectance using calibration standards with known optical properties and (2) implementing an iterative fitting routine based on the LUT. In certain embodiments, a nonlinear optimization fitting routine may be used to fit the reflectance spectra. Such a routine may comprise (1) constraining the reduced scattering coefficient to the form μs′(λ)=μs′(λ0)·(λ/λ0)−B where λ0=630 nm, (2) calculating an absorption coefficient using the absorption crosssections σ_{Hb} and σ_{HbO2} as μₐ=[Hb]*(α_{σHbO2}+(1−α)σ_{Hb})+λ, where α is the oxygen saturation of the tissue, Hb is the total hemoglobin concentration of the tissue, and λ is adsorption coefficient of a chromophore (e.g., melanin, beta-carotene, a dye (e.g., indocyanine green)). In certain embodiments, it may be assumed that the absorption in the visible range to be due to oxy- and deoxy-hemoglobin. In certain embodiments, depending on the type of tissue sampled and the wavelength range of interest, the expression for μa(λ) can be modified to include the absorption cross-sections of other absorbing chromophores. In certain embodiments, the look-up algorithm may be used to determine the tissue parameters displayed by the software interface of the systems of the present invention. For example, in certain embodiments, laser excitation at 337 nm generates fluorescence from the metabolic coenzyme NADH and collagen, while laser excitation at 400 nm generates fluorescence from FAD. Also, in certain embodiments, white light, such as light from xenon flashlamps, may be used to collect elastic scattering spectra. Both NADH and FAD are associated with tissue metabolism and can be used to determine the tissue redox ratio. In certain embodiments, elastic scattering spectra can be fit to a diffusion theory model to extract the blood oxygen saturation, blood concentration, melanin concentration, and tissue scattering parameters.

Coupling

The MMS probe has 2 input connections: one for the 830-nm Raman laser and one port for both the N2 laser and the Xenon lamp. Raman and LIFS/DRS ports are separated as near infrared (NIR) light for the Raman modality has different optical design requirements (fiber material, filters, transmission, etc.) than the ultraviolet and visible wavelengths used in DRS and LIFS.

The white light and laser pulses (DRS and LIFS modes) are coupled into optical fibers and guided into a 3×1 fiber optic switch (e.g., FSM-13, Piezosystems Jena, Germany). The switch is a microelectromechanical (MEMS) device, which uses microprisms to control and open different optical ports to ensure that the 377-nm laser light and broadband Xenon light are separated and coupled sequentially into the MMS probe without any overlap. The switch is controlled via transistor logic (TTL) pulse trains initiated within the custom software. Light from the switch's output is passed to the dual LIFS/DRS input port of the MMS probe via a subminiature version A (SMA) nipple fitting; roughly 30% loss in signal is measured due to the optical switch and SMA fitting. The 830-nm Raman laser light is delivered without the optical switch and is triggered after the LIFS and DRS pulses.

Photodetection

The MMS detection hardware consists of components optimized for visible (LIFS and DRS) and NIR (RS) detection.

Reflectance and Fluorescence

The LIFS/DRS spectral system comprises a interline CCD camera (e.g., CoolSNAP HQ, Princeton Instruments, Trenton, N.J.) cooled to −30° C. For each of the LIFS and DRS pulses, the CCD is gated at 50 μs. The distal ends of the two DRS/LIFS fibers are aligned with the vertical axis of the spectrograph (e.g., SpectraPro 2150i, Princeton Instruments, Trenton, N.J.) using software provided by the manufacturer (e.g., WinSpec, Princeton Instruments, Trenton, N.J.). A 150 grooves/mm grating, blazed at 500 nm, is used in order to capture the entire visible spectrum needed for LIFS (385-650 nm) and DRS (375-700 nm). A slit width of 200 μm is used. To improve the SNR, we bin every three pixels for a spectral dispersion of 0.78 nm/pixel and a resulting spectral resolution FWHM of 5.6 pixels (4.32 nm).

Raman

The Raman system consists of a 1024×1024 camera (e.g, IMG, Finger Lakes Instrumentation, NY) cooled to −30°266 C which is coupled to an f/1.8 spectrograph (e.g., Holospec, Kaiser Optical Systems, MI) utilizing a low-frequency Stokes grating for 830 nm excitation. The longer Raman excitation wave length reduces tissue autofluorescence by a factor of four compared to 785 nm excitation. A custom 200 μm width slit was installed. This slit width was selected as it strikes an effective balance between ensuring sufficient resolution for spectral analysis and signal strength. The measured spectral dispersion is 1.79 cm⁻¹ per pixel and the spectral resolution is a FWHM of 13.43 cm⁻¹.

Data Acquisition Hardware and Software

Custom software has been written in LabVIEW (National Instruments, Austin, Tex.) for single-click operation of the entire MMS system. The software executes MMS data collection by sequentially capturing DRS, LIFS, and RS spectra. For the DRS and LIFS modalities, the sources are triggered for data acquisition via TTL pulses provided by a timer counter board (NI 2121, National Instruments, Austin, Tex.) while for RS this same timer-counter board triggers the mechanical shutter to open (as the diode laser is a continuous source and therefore always on). The DRS and LIFS camera is controlled by a PCI card (e.g., PCI-6602, National Instruments, Austin, Tex.) and operated, in part, by pre-written software (e.g., R3 Software, Princeton, N.J.). At the basic software architecture level, the Raman instrument components (laser and camera) are controlled via drivers written in C++ and incorporated into a MATLAB code; however, these codes and drivers are called and user inputs implemented within LabVIEW. Spectra are displayed for instant user feedback via onboard binning and background subtraction. Finally, for the DRS and LIFS modalities, an extra step is required for optical switch operation.

Calibration

To achieve day-to-day consistency and accurate spectral measurements, calibration procedures must be performed. The Raman and LIFS/DRS wavelength calibrations are performed by measuring known spectral lines from a solid 4-acetamidophenol (Tylenol) capsule and a mercury-argon pencil lamp (Hg-1, Ocean Optics, FL), respectively. To account for how the quantum efficiency of the detectors alter the fluorescence and Raman spectra (system response), the spectrum of a calibrated Tungsten light source (e.g., LS1-Cal, Ocean Optics, FL) shone onto a 20% reflectance standard is recorded; the system response is not required for the DRS measurement as it is inherently normalized. To account for white light source day-to-day variations, the reflectance amplitude is measured by recording the spectrum of a solid titanium dioxide standard.

This step ensures that all reflectance measurements are calibrated to the LUT model before extraction of optical properties. Background calibration, which accounts for stray light and dark current, is performed for all three modalities by taking spectra with the lights off. With external triggering, the shot-to-shot variation from the N2 laser was measured at 12%. Therefore, to account for these fluctuations, a beam splitter was installed to create a power measurement arm and each fluorescence spectra is then normalized by this measured power (e.g., 3A-P, Ophir Optics, Israel). These calibration procedures were performed prior to every clinical data collection day (to account for day-to-day variations) and also whenever any optical alignment took place such as distal end reinsertion into a spectrometer, slit width adjustment, and camera or lens alignment.

The output energy of the N2 laser is measured at 6.5 μJ, which is considerably lower than the maximum permissible levels (53 μJ) of a Class 1 device. The mechanical shutter, which blocks the 830-nm laser diode laser, opens when triggered through the software, closes immediately after the acquisition and remains closed until the next acquisition.

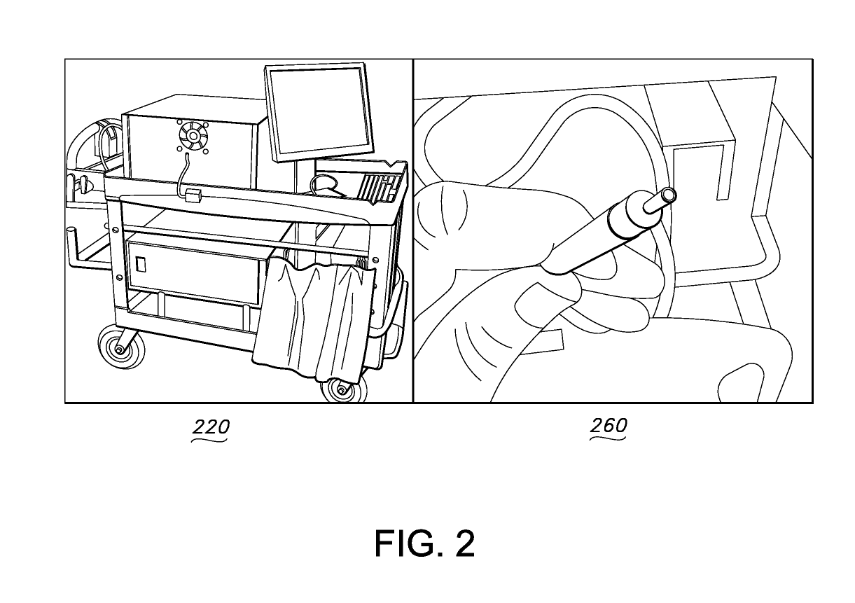

For clinical portability, all MMS components are mounted to a two-level utility cart (e.g., 4546-10, Rubbermaid, Winchester, Va.) as shown in FIG. 2. The utility cart 220 was specially outfitted with 6-in. pneumatic caster wheels to prevent vibration and increase transportation ease. To ensure electrical line isolation and prevent electrical damage, all powered MMS components (spectrometers, cameras, etc.) are connected to the main power via an isolation transformer power conditioner (e.g., IS250, Tripplite, Chicago, Ill.). Furthermore, in the event of main power loss and to allow the system to be transported between clinical rooms, the MMS system is also connected to a battery supply (e.g., CP1500AVR, CyberPower, Shakopee, Minn.) which provides approximately 10 min of external power. FIG. 2 also illustrates an embodiment of a probe 260 according to the present disclosure.

Spectral Modeling and Analysis

Reflectance

The diffusion approximation to the radiative transport equation, which is most commonly used in order to extract optical property information from reflectance spectra, is only valid when the separation between the excitation fiber (source) and collection fibers (detector) are less than the mean free path and in absorption-dominated tissues (high albedo absorption scattering). MMS work conducted by other researchers, who studied atherosclerosis, used this diffusion approximation to calculate reflectance using their MMS probe. However, the diffusion approximation is not valid for our application as skin cancers originate in shallow cutaneous layers, which requires relatively short source-detector separations to confine the light only to these shallow layers, and the lesions of interest (i.e., dysplasia) can have low albedo. As the diffusion approximation is not valid, we use a look-up table (LUT) inverse model approach that is appropriate for our conditions (short source-detector separation and low albedo) and accurately measures tissue optical properties such as the reduced scattering coefficient, μ′s, and absorption coefficient, μₐ. It has been shown by previous researchers that the diffuse reflectance will be the same for any combination of μs and g that result in the same μ′s. We have previously shown in our laboratory—by performing detailed parametric studies—that the extracted absorption and reduced scattering coefficient values have less than 10% error when the anisotropy is greater than 0.7 when using the LUT method. The LUT is essentially a database of reflectance values across a range of scattering and absorption values. The database is generated by measuring reflectance spectra from a matrix tissue simulating phantoms with known optical properties and then interpolating between these values to generate a topography in R, μs, and μ′s space. Any reflectance spectra, obtained from a sample with unknown optical properties, can be fit to this database in order to determine its optical properties. The tissue phantoms are created by using polystyrene beads with nominal 1 μm diameter and 2.6% solids by volume (e.g., Polysciences, Warrington, Pa.) and black India ink (e.g., Speedball, Statesville, N.C.) as the scattering and absorption media, respectively. Mie theory was used to calculate the μ′s of the polystyrene beads (and therefore the amount of volume to add for a desired μ′s) and a spectrophotometer (e.g., DU720, Beckman Coulter, CA) to measure the μₐ of a stock India ink solution. In total, 21 phantoms were used to generate the LUT, spanning physiological relevant values of μ′s (0.44-4.74 mm⁻¹) and μₐ (0-2.5 mm⁻¹). Raw spectra were then collected by the probe and reflectance spectra calculated using the following equation:

$${R}_{diffuse} ( λ ) = \frac{{I}_{sample} ( λ ) - {I}_{background} ( λ )}{[ {I}_{standard} ( λ ) - {I}_{background} ( λ ) ] \times 100 / {R}_{standard}} , \qquad ( 1 )$$

where Iₛₐₘₚₗₑ(λ) is the raw spectrum from the phantom, I_{background}(λ) is the background spectrum, I_{standard}(λ) is the spectralon standard spectrum and 100/R_{standard} is a factor used to account for the calibrated reflectance level of the spectralon standard (throughout this paper all results were obtained with a 20% spectralon reflectance standard). Spectra are presented in terms of wavelength by performing the wavelength calibration procedure discussed above. Validation of the LUT is discussed below. This approach follows previous work conducted in our laboratory and the successful application of the LUT approach for skin cancer diagnosis.

A nonlinear optimization fitting routine is employed to minimize the difference (×2) between the database LUT reflectance spectra and the measured reflectance spectra between 400 and 650 nm. For the LUT validation (see Sec. IV A), the reduced scattering coefficient and absorption coefficient are constrained to the following forms:

$$\begin{matrix}{μ}_{s}^{′} ( λ ) = {μ}_{s}^{′} ( {λ}_{0} ) {( \frac{λ}{{λ}_{0}} )}^{- B} , & ( 2 ) \\ {μ}_{a} ( λ ) = \frac{2.303 yA ( λ ) [ absorber ]}{L} , & ( 3 )\end{matrix}$$

where λ₀ is 630 nm, A(λ) is the absorbance spectra of the dye when measured using a spectrophotometer, L is the path length of the spectrophotometer measurement, [absorber] is the concentration of absorber used for the spectrophotometer measurement, and y is a scaling factor to account for the dilution of the dye solution used in the spectrophotometer. For the validation study, the fitting outputs are μ′s(λ₀), B, and [absorber], from which μ′s(λ) and μₐ(λ) can be calculated. For clinical fitting (see Sec. IV B), the reduced scattering coefficient is constrained in the same fashion (Eq. (2)), however, the physiological absorption coefficient is calculated using the following equations described by van Veen:

$$\begin{matrix}{μ}_{a ( blood )} ( λ ) = 150 υ [ {αɛ}_{{HbO}_{2}} ( λ ) + ( 1 - α ) {ɛ}_{Hb} ( λ ) ] , & ( 4 ) \\ {C}_{pack} = [ \frac{1 - {e}^{( - 2 {μ}_{a ( bl )} ( λ ) {D}_{vessel} )}}{2 {μ}_{a ( bl )} ( λ ) {D}_{vessel}} ] , & ( 5 ) \\ {μ}_{a ( tissue )} ( λ ) = {C}_{pack} {υμ}_{a ( bl )} ( λ ) + [ mel ] {ɛ}_{mel} ( λ ) , & ( 6 )\end{matrix}$$

where ν is the blood volume fraction sampled by the light (assuming a hemoglobin concentration of 150 mg/ml in the bloodstream), α is the oxygen saturation (ratio of HbO2 to total Hb), ε_{HbO2}(λ), ç_{Hb}(λ) and εₘₑₗ(λ) are the extinction coefficients of oxygenated hemoglobin, deoxygenated hemoglobin, and melanin, respectively, Dᵥₑₛₛₑₗ is the mean vessel diameter and [mel] is the concentration of melanin. These equations are used to account for the inhomogeneous distribution of blood vessels in tissue. For clinical data, the fit outputs are μ′s(λ₀), B, [mel], ν, α, and Dᵥₑₛₛₑₗ.

Fluorescence

Fluorescence spectra are first background corrected by subtracting a dark spectrum (lights off, probe pointing upwards) from the raw spectrum. Next, a wavelength calibration is performed by using a peak fitting algorithm to find the pixel locations of HgAr lines, fitting a 3rd order polynomial (as 4 strong lines are seen in the visible) from these pixel locations to the known wavelengths of these lines and then converting the entire pixel array to wavelength space. The intensity calibration is performed by scaling the measured blackbody spectrum to the measured values provided by the manufacturer; the measured spectrum will be altered due to the wavelength dependence of the detector's quantum efficiency and this step is necessary in order to correct for this instrument response.

The turbid nature of raw tissue alters the fluorescence signal such that the measured fluorescence spectral shape is altered and its intensity attenuated. Therefore, the intrinsic fluorescence—the true endogenous fluorescence without scattering or absorption distortion—must be calculated in order to accurately model physiological fluorescence. The intrinsic fluorescence is calculated by using the photon migration model of Zhang et al., which uses the measured reflectance of the sample (with a particular μₐ. and μ′s and probe specific parameters in order to correct the fluorescence

$$IF ( λ ) = \frac{F ( λ )}{\frac{R ( {λ}_{x} )}{{μ}_{s} ( {λ}_{x} ) {IR}_{0} ( {λ}_{x} )} \sqrt{\frac{{R}_{0} ( {λ}_{x} ) {R}_{0} ( λ )}{ɛ ( {λ}_{x} ) ɛ ( λ )}} [ \frac{R ( λ )}{{R}_{0} ( λ )} + ɛ ( λ ) ]} , \qquad ( 7 )$$

where λx is the excitation wavelength, μₛ(λₛ) is the value of the scattering coefficient at the excitation wavelength, IF(λ) is the intrinsic fluorescence spectrum, F(λ) is the measured fluorescence spectrum, R(λ) is the measured reflectance spectrum, R(λ) is the value of the measured reflectance at the excitation wavelength, R0(λ) is the measured reflectance spectrum for no absorption, and R₀(λₓ) is the value of the reflectance at the excitation wavelength for no absorption. F(λ) and R(λ) are directly measured via experiment and R(λx) is a constant determined from the R(λ) spectrum. R₀(λ) is calculated by first fitting the measured spectrum, R(λ), to the LUT to obtain μₐ and μ′s. The corresponding reflectance spectrum with this μ′ₛ is then numerically evaluated via two-dimensional interpolation of the LUT space by constraining μₐ=0. Additionally, E(λ) is calculated using e=εβ−1, where β=S(1−g) for a probe specific constant, S, and the anisotropy, g; ε(λₓ) is the value of E(λ) at the excitation wavelength. The last remaining term, l, is also a probe specific constant.

As discussed above, S and l are probe specific parameters and they are determined empirically. These were calculated as follows. First, the fluorescence spectra of 0.26 μM molar Stilbene (Exciton, OH) dissolved in ethanol was measured. Stilbene-420 was chosen as its peak emission wavelength is similar to that of NADH. The heavily diluted 0.2 μM solution was chosen in order to ensure that the Stilbene itself was not contributing to the scattering signal while guaranteeing sufficient fluorescence SNR. Without any scattering or absorption, this spectra corresponds to IF(λ). Next, a spectra was taken with polystyrene beads added to the 0.26 μM Stilbene solution to create a solution with μ's=1.5 mm⁻¹. As this spectra is only influenced by scattering (no absorption), it is equal to R₀(λ) and from which R₀(λₓ) can be determined. In sequential order, 4 volumes of red dye were added to the solution to create solutions with μₐ values of 0.29, 0.57, 0.86, and 1.14 mm⁻¹. These spectra corresponded to four different R(λ). An optimization routine was written to calculate S and l by minimizing the difference between the four IF(λ) spectra when calculated using Eq. (2). The values of S and l, specific to our particular MMS probe, are 1.83 and 0.163.

In tissue, 337 nm excites both NADH and collagen. We assume that the measured intrinsic fluorescence is a linear combination of fluorescence from NADH and collagen, which can be expressed mathematically as

IF(λ)=A₁IF_{NADH}(λ)+A₂IF_{NADH}(λ), (8)

where IF_{NADH}(λ) and IF_{collagen}(λ) are the intrinsic spectra of NADH and collagen, respectively, and χ_{nadh}=A1/(A1+A2) and χ_{collagen}=A2/(A1+A2) represent the relative concentrations of NADH and collagen, respectively. The values of A₁ and A₂ are determined by a fitting routine.

Raman

Raman spectra is collected in the “fingerprint” region (˜400-1800 cm−1), because it is a rich source of Raman bio-markers useful for skin properties and skin cancer diagnosis. The first processing step involves background subtraction whereby a dark spectrum is subtracted from the raw spectrum. Second, the wavelength and intensity calibrations are performed in a very similar manner as described for the fluorescence spectra; the only difference is that the peak fitting algorithm is used to find Tylenol pixel locations (instead of HgAr lines as needed for the fluorescence). Raman incident laser light can cause tissue autofluorescence, which has practical implications as the fluorescence swamps the Raman signal. This autofluorescence is most likely due to the morphological make-up of the epidermis, which absorbs light up to 1000 nm and can re-emit it as fluorescence. Tissue autofluorescence is removed by fitting the Raman spectra to a 5th order polynomial and subtracting the fit from the raw spectrum, revealing the desired endogenous Raman signals.

MMS System and Probe Performance

LUT Validation

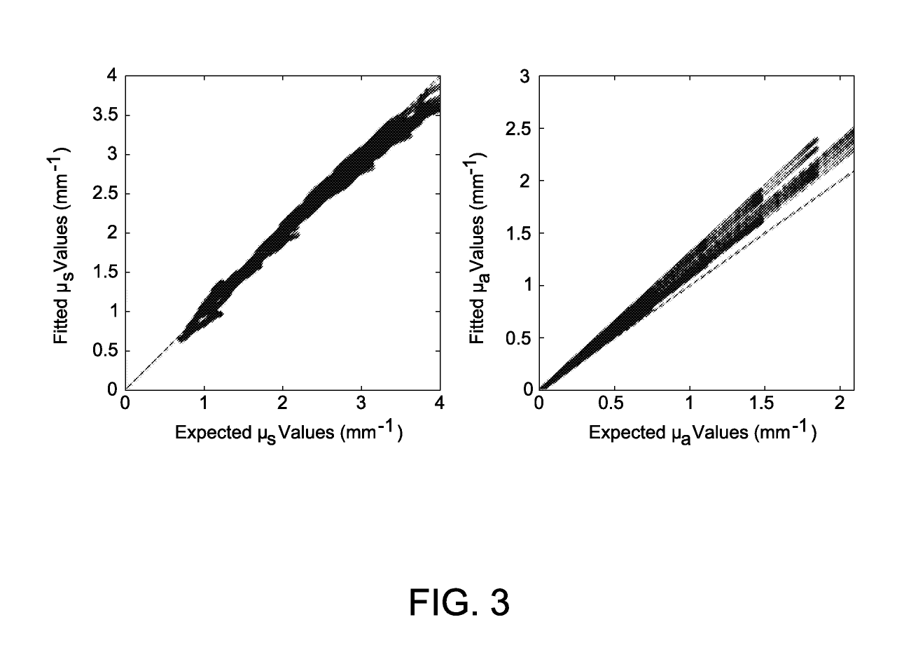

The LUT was validated by fitting the LUT topography to spectra obtained from 47 validation phantoms with known optical properties. The validation phantoms were fabricated by using the polystyrene beads and colored food dye (red, blue, and green) to simulate scattering and absorption, respectively. These phantoms spanned ranges of 0.72-4.31 mm⁻¹ and 0-2.42 mm⁻¹ for μ′s and μₐ, respectively, which covered approximately 90% of the LUT surface. For each validation phantom, spectra were averaged across 5 measurements. Using the LUT fitting approach described above, the optical properties were extracted with normalized root-mean-square errors of 7.19% and 9.81% for μ′s and μₐ, respectively, as shown in FIGS. 3(a) and 3(b); for a particular optical property, the errors were calculated by averaging across all wavelength and all 47 phantoms.

Raman In Vivo Performance

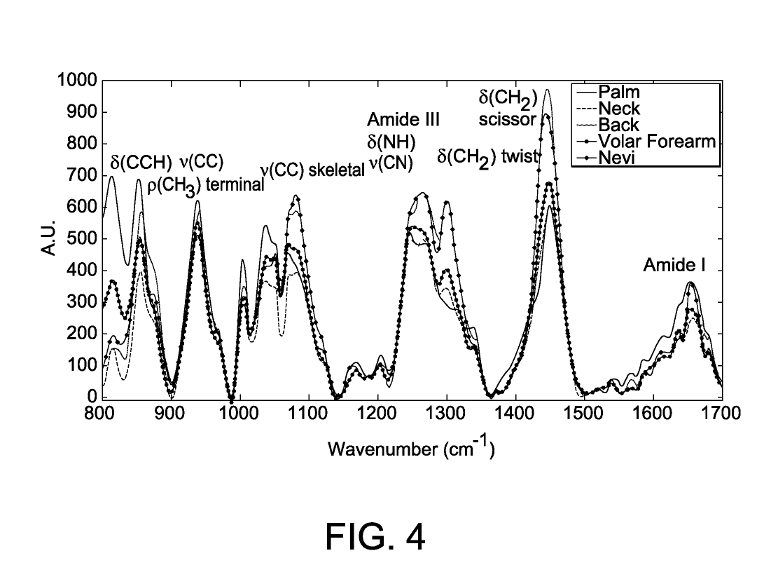

To demonstrate the Raman performance, in vivo Raman spectra were obtained at different locations on the body of a healthy Caucasian male volunteer, FIG. 4. Each location was measured 5 times. Tissue autofluorescence was removed using the polynomial fitting procedure described previously. Encouragingly, bands identified by previous researchers as being important for skin cancer diagnosis—the Amide I, Amide III, and CH₂ scissor—show very good signal to noise ratios.

MMS Clinical Performance

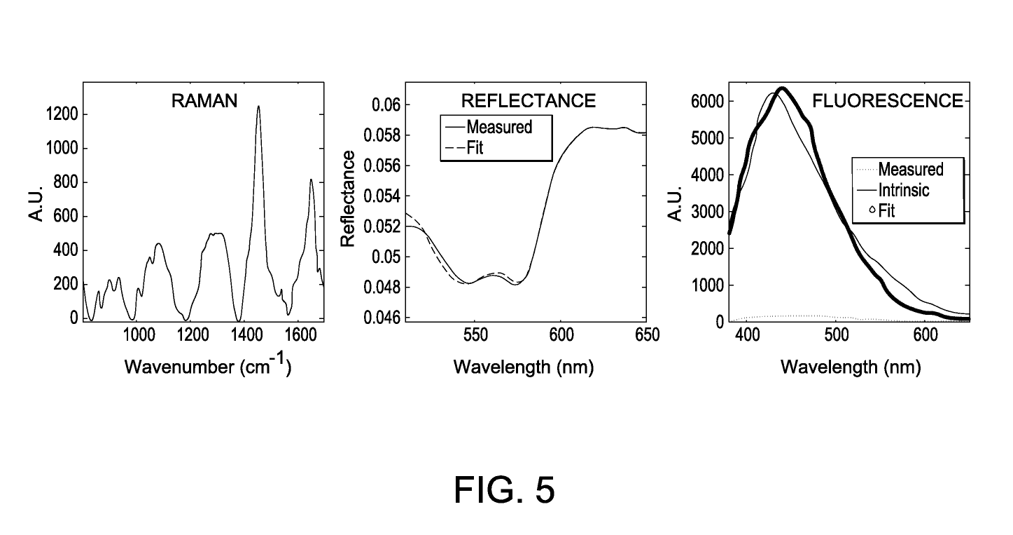

The MMS system is currently being used for clinical testing for the detection of non-melanoma and melanoma skin cancers. Clinical data acquisition times are roughly 4.5 s in total comprising a 4 s Raman exposure and the 3 Xenon flashes, N2 laser pulse, and optical switching making up the remaining 500 μs. The clinical data acquisition procedure was as follows: (1) Dermatologist identified suspicious lesion, (2) 3 repeat measurements made on each lesion, (3) 3 repeat measurements of corresponding normal skin as close to the lesion as possible, and (4) lesion is biopsied and lesions classified using histopathology. Sample MMS spectra are presented in FIG. 5 for a basal cell carcinoma. Fitting to Eqs. (2)-(6) and (8) above gives physiological quantities consistent with a non-melanoma skin cancer clinical study previously conducted by our group.

As our final long-term goal is to apply this technology in the clinic, two major evaluations would be the specificity/sensitivity of the instrument and its repeatability. As the clinical data collection is ongoing, specificity/sensitivity data are not presented at this stage. However, bench top and clinical data have been collected to quantify the repeatability of the MMS system. In order to quantify the repeatability of the system, each modality was characterized separately. For Raman, the standard deviation as a percentage of the mean for 50 peak intensity measurements of a major Tylenol peak (at ˜1324 cm⁻¹) was calculated to be 1.6% and the variation in Scissor band peak (˜1450 cm⁻¹) intensity from 5 measurements (from section entitled “Raman in vivo performance,” below) was calculated to be 6%. It is expected that the in vivo measurement displays greater variation than the bench top measurement due to patient movement. For DRS, the repeatability was measured to be 3.2% for μ′s and 3.4% for μₐ. These values were determined by averaging across 3 sets of 20 measurements each (total of 60 measurements) for the reflectance values at maximum absorption and at 630 nm for the 3 most highly absorbing and highly scattering liquid phantoms (blue, green, and red with μ′s (630 nm)=4.31 and μₐ=2.32 mm⁻¹). From 5 measurements of the volar forearm, the variation was 7.0% and 8.2% for values at 630 nm and 417 nm (Hemoglobin Soret band peak). For LIFS, the repeatability was assessed by calculating the variance in 100 measurements of the fluorescent peak of a heavily diluted Coumarin sample (Exciton, OH) at 337 nm excitation. Without accounting for the laser fluctuation, the variance is 17% and when normalizing the signal by the power arm measurement (see “Calibration” section) this variance dropped to 4%, which clearly demonstrates the need for the power arm measurement in order to account for laser fluctuations.

The addition of an absorber to a phantom involves extra pipetting steps in order to complete the spectrophotometer measurement, which is not needed for a purely scattering phantom. It is proposed that the lower extraction accuracy of μₐ compared to μ′ₛ is due to the extra steps which represent more sources of measurement error due to instrument resolution and calibration (spectrophotometer, pipettes) and also human error. At this stage, we have no definitive explanation for the μₐ trend of increasing divergence between fitted and expected values (as seen in FIG. 3) with increasing μₐ values. However, the extra μa measurement steps could offer an explanation as equipment error (spectrophotometer, pipettes) could be proportional to volume or concentration, which would result in the systemic divergence observed.

There is a clear discrepancy between the intrinsic fluorescence fit (used to determine the collagen and NADH relative concentrations) and the extracted measured intrinsic fluorescence, FIG. 5. This discrepancy is primarily due to the fact that our intrinsic fluorescence model does not fully capture the physics. We assume linear superposition of the two fluorophores, which implies that their measured fluorescence is independent of each other. In reality, these fluorescence signals are coupled as NADH has non-negligible absorption within the visible and will therefore absorb some of collagen's fluorescence emission—the opposite situation will also occur (collagen absorbing some of NADH's fluorescence). Therefore, the measured flux from both NADH and collagen fluorescence will be attenuated due to this fluorescence emission-absorption overlap and will spectrally manifest itself as attenuated signal and a slightly different peak location. A more accurate model would include second-order, nonlinear terms to describe this fluorescence emission-absorption overlap; however, previous researchers have successfully applied this linear superposition approach for clinical diagnosis as it still does a very good job of capturing the endogenous fluorescence physics. It will be apparent to those skilled in the art that various modifications and variations can be made to the disclosed systems and methods for multi-modal characterizing biological tissue. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

What is claimed is:

1. A fiber-optic probe for multi-modal characterization of a tissue comprising:
a first group of fibers associated with a first modality of light, the first group of fibers comprising a first light delivery fiber and a first light collection fiber;
a second group of fibers associated with at least a second modality of light, the second group of fibers comprising a second light delivery fiber and a second light collection fiber;
a longpass filter positioned distal to the first group of fibers; and
a lens positioned distal to the filter;
wherein the second group of fibers bypasses the filter.

2. The probe of claim 1, wherein the second group of fibers bypasses the filter by extending through an aperture in the filter.

3. The probe of claim 1, further comprising an inner wall of the probe, wherein the filter is sized to allow the second group of fibers to bypass the filter by fitting between the filter and the inner wall of the probe.

4. The probe of claim 1, wherein:
the second light delivery fiber emits light onto an illuminated region of the tissue, and
the lens comprises a wedge-shaped portion and a reflective coating on the wedge-shaped portion, the wedge-shaped portion and the reflective coating configured to direct light emitted from the first light delivery fiber toward the illuminated region.

5. The probe of claim 1, wherein the lens is cut at an angle to form a wedge-shaped portion of the lens.

6. The probe of claim 1, wherein:
the filter and the lens each have a peripheral edge, the peripheral edge of the filter being aligned with the peripheral edge of the lens, and
the second group of fibers extends to a distal end of the probe and bypasses the filter and the lens by abutting the peripheral edge of the lens and the filter.

7. The probe of claim 6, wherein the second group of fibers contacts a surface of the tissue when in use.

8. The probe of claim 6, wherein the peripheral edge of the filter and the lens forms a horizontal cylindrical segment of the filter and the lens.

9. The probe of claim 6 wherein the peripheral edge of the filter and the lens comprises a notch in the filter and the lens.

10. The probe of claim 1, wherein the first light delivery fiber is centrally located at a distal end of the probe.

11. The probe of claim 1, wherein the first light delivery fiber is concentrically surrounded by (a) the first light collection fiber and (b) the second group of fibers.

12. The probe of claim 1, wherein the second group of fibers bypasses the lens.

13. The probe of claim 2, wherein the second group of fibers bypasses the lens by extending through an aperture in the lens.

14. The probe of claim 3, wherein the lens has an anti-reflective coating.

15. The probe of claim 14, wherein the anti-reflective coating of the lens is capable of reducing reflections that interfere with a diffuse reflectance modality of the probe.

16. A system for multi-modal characterization of a tissue comprising:
the fiber-optic probe of claim 1; and
a computer processor in operative communication with the probe and configured to use a lookup table inverse model to correct distortion of a source-detector geometry of the second group of fibers.

17. The probe of claim 1, wherein the lens comprises a plano convex back portion and a flat front portion.

18. The probe of claim 17, wherein the plano convex back portion comprises sapphire.

19. The probe of claim 17, wherein the flat front portion comprises magnesium fluoride.

20. A method for multi-modal characterization of biological tissue, comprising:
collecting first light emitted from a biological tissue through a first transmission medium of a fiber-optic probe, the first transmission medium including a lens;
collecting second light emitted from the biological tissue through a second transmission medium, the second transmission medium bypassing the lens; and
processing the collected first and second light to determine a characteristic of the biological tissue.

21. The method of claim 20, wherein the collected first light is processed, at least in part, using Raman spectroscopy.

22. The method of claim 20, wherein the collected second light is processed, at least in part, using diffuse reflectance spectroscopy.

23. The method of claim 20, wherein the collected second light is processed, at least in part, using laser-induced fluorescence spectroscopy.

24. A method for multi-modal characterization of biological tissue, comprising:
delivering, via a first optical fiber through a first transmission medium, first light from a first light source onto a biological tissue;
collecting, via a second optical fiber through the first transmission medium, light emitted from the biological tissue in response to the first light;
delivering, via a third optical fiber through a second transmission medium, second light from a second light source onto the biological tissue;
collecting, via a fourth optical fiber through the second transmission medium, light emitted from the biological tissue in response to the second light; and
processing the light collected by the second and fourth optical fibers to determine a characteristic of the biological tissue.

25. The method of claim 24, wherein the first transmission medium includes at least one filter and a lens.

26. The method of claim 25, wherein the second transmission medium bypasses the at least one filter and the lens.

27. The method of claim 24, further comprising:
delivering, via the third optical fiber through the second transmission medium, third light from a third light source onto the biological tissue;
collecting, via a fifth optical fiber through the second transmission medium, light emitted from the biological tissue in response to the third light.

28. The method of claim 24, wherein processing the light collected by the second and fourth optical fibers comprises using a lookup table inverse model to correct distortion of a source-detector geometry of the second group of fibers.
